(19) United States
(12) Patent Application Publication
(10) Pub. No.: US 2017/0304451 A1
(43) Pub. Date: Oct. 26, 2017

(54) PARENTERAL COMPOSITIONS OF BENDAMUSTINE

(71) Applicant: HETERO RESEARCH FOUNDATION, HYDERABAD (IN)

(72) Inventors: BANDI PARTHASARADHI REDDY, ANDHRA PRADESH (IN); PODILI KHADGAPATHI, ANDHRA PRADESH (IN); BANDARI SREEDHAR, ANDHRA PRADESH (IN)

(21) Appl. No.: 15/645,237

(22) Filed: Jul. 10, 2017

Related U.S. Application Data
(62) Division of application No. 15/203,999, filed on Jul. 7, 2016.
Application No. 15/203,999 is a continuation-in-part of application No. PCT/IN2015/000015, filed on Jan. 12, 2015.

(30) Foreign Application Priority Data
Jan. 13, 2014 (IN) 151/CHE/2014

(51) Int. Cl. A61K 47/22; A61K 31/4184; A61K 9/08; A61K 47/10; A61K 9/00

(52) U.S. Cl. CPC A61K 47/22; A61K 47/10; A61K 9/0019; A61K 9/08; A61K 31/4184

(57) ABSTRACT

Described herein are parenteral compositions of bendamustine. More particularly, parenteral compositions of bendamustine are in the form of solution.

### PRIORITY

This patent application is a divisional of U.S. application Ser. No. 15/203,999 filed on Jul. 7, 2016, which is a continuation in part of PCT/IN2015/000015 filed on Jan. 12, 2015, which claims priority to Indian patent application number 151/CHE/2014, filed on Jan. 13, 2014, the contents of which are incorporated by reference herein in their entirety.

### FIELD OF THE INVENTION

The present invention relates to parenteral compositions of bendamustine and processes for preparation thereof.

### BACKGROUND

Chemically bendamustine hydrochloride is 1H-benzimidazole-2-butanoic acid, 5-[bis(2-chloroethyl)amino]-1 methyl-, mono hydrochloride. Its empirical formula is C₁₆H₂₁C₁₂N₃O₂. HCl, with a structural formula as follows:

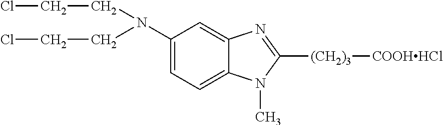

In the United States, bendamustine is available as a powder for IV infusion in the strengths of 100 mg/vial and 25 mg/vial, and also as a solution for IV infusion in the strengths of 180 mg/2ml (90 mg/ml) and 45 mg/0.5 ml (90 mg/ml), with a trade name Treanda® by Cephalon.

U.S. Pat. No. 8,436,190 discloses lyophilized pharmaceutical compositions of bendamustine.

U.S. Pat. No 8,344,006 discloses stable, non-aqueous liquid formulations comprising bendamustine solubilized in dimethylacetamide and propylene glycol.

U.S. Pat. No. 8,609,707 discloses stable, non-aqueous liquid compositions comprising bendamustine, antioxidant and a fluid comprising polyethylene glycol, propylene glycol.

There still exists a need for alternative solvent systems to prepare stable compositions of bendamustine.

Inventors of the present invention have developed stable ready to use parenteral compositions of bendamustine using alternative solvent systems.

### SUMMARY OF THE INVENTION

The present invention relates to parenteral compositions of bendamustine.

One embodiment of the present invention relates to a ready to use parenteral composition comprising: a) bendamustine or its pharmaceutically acceptable salt and b) N-methyl-2-pyrrolidone as a solvent.

Another embodiment of the present invention relates to a ready to use parenteral composition comprising bendamustine or its pharmaceutically acceptable salt, diethylene glycol monoethyl ether, and a polar solvent selected from N-methyl-2-pyrrolidone (NMP) and polyethylene glycol.

One another embodiment of the present invention relates to a ready to use parenteral composition comprising per each ml of composition:
a) 90 mg of bendamustine hydrochloride,
b) 0.1 to 0.5 ml diethylene glycol monoethyl ether, and
c) a polar solvent selected from N-methyl-2-pyrrolidone (NMP) and polyethylene glycol.

Also included in the present invention is the use of bendamustine compositions for the treatment of chronic lymphocytic leukemia and B-cell non-Hodgkin lymphoma.

### DETAILED DESCRIPTION OF THE INVENTION

The present invention relates to parenteral compositions of bendamustine. More particularly, the present invention includes ready to use parenteral compositions of bendamustine in the form of solutions.

The term “active ingredient” or “active agent” or “drug” used interchangeably, is defined to mean active drug (e.g. bendamustine), that induces a desired pharmacological or physiological effect.

The term “bendamustine” as used herein includes bendamustine in the form of a free base, a pharmaceutically acceptable salt thereof, amorphous bendamustine, crystalline bendamustine or any isomer, derivative, hydrate, solvate, or prodrug or combinations thereof. Preferably, bendamustine is in the form of the hydrochloride salt. More preferably, the bendamustine salt is bendamustine hydrochloride monohydrate.

The term “pharmaceutically acceptable” as used herein means that which is useful in preparing a pharmaceutical composition that is generally safe and non-toxic.

The term “excipients” as used herein means a component of a pharmaceutical product that is not an active ingredient. The excipients that are useful in preparing a pharmaceutical composition are generally safe and non-toxic.

The term “parenteral” as used herein means administration through intravenous, intramuscular, subcutaneous, intracutaneous, intra-articular, or intrathecal routes of administration, preferably, intravenous.

As used in the specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

The term “ready to use composition” as used herein refers to a composition which avoids reconstitution and may require dilution with a suitable diluent before administration to the patient.

The term “solvent” refers to an ingredient used for dissolving an active ingredient. Exemplary polar solvents include N-methyl-2-pyrrolidone, 1,3-dimethyl-2-imidazolidinone, dimethylacetamide, acetone, tetrahydrofuran, 1,4-dioxane, acetonitrile, dimethyl formamide, propylene carbonate, alkyl alcohols, ethylene glycol, propylene glycol, butylene glycol, glycerin, polysorbates, polyalkylene glycols such as polyethylene glycol, and primary amides and combinations thereof. Preferably, the solvent is N-methyl-2-pyrrolidone, polyethylene glycol, or a combination thereof

N-Methyl-2-pyrrolidone as used in the present invention is synonymously referred as 1-methyl-2-pyrrodinone, 1-methyl-5-pyrrodinone, N-methyl-a-pyrrolidinone, N-methyl-g-butyrolactam, Nmethyl-2-pyrrolidinone, 1-methylazacyclopentan-2-one, N methylpyrrolidonum, MP, NMP, Pharmasolve™, m-Pyrol.

Diethylene glycol monoethyl ether marketed by Gattefosse under the brand name Transcutol® is optionally used as a co-solvent in an amount of 0.01 ml to 1 ml preferably, 0.1 to 0.5 ml.

One embodiment of the present invention relates to a ready to use parenteral composition comprising: a) bendamustine or its pharmaceutically acceptable salt and b) N-methyl-2-pyrrolidone as a solvent. In another embodiment, a ready to use parenteral composition consists essentially of, or consists of a) bendamustine or its pharmaceutically acceptable salt and b) N-methyl-2-pyrrolidone as a solvent.

One embodiment of the present invention relates to a ready to use parenteral composition comprising bendamustine or its pharmaceutically acceptable salt, diethylene glycol monoethyl ether, and a polar solvent selected from N-methyl-2-pyrrolidone (NMP), and polyethylene glycol. In another embodiment, a ready to use parenteral composition consists essentially of, or consists of, bendamustine or its pharmaceutically acceptable salt, diethylene glycol monoethyl ether, and a polar solvent selected from N-methyl-2-pyrrolidone (NMP), and polyethylene glycol.

The composition according to the present invention is in the form of a solution, suspension, emulsion or lyophilized powder. Preferably, the composition is in the form of a solution.

Another embodiment of the present invention relates to a ready to use parenteral composition comprising about 25 mg/ml to about 100 mg/ml of bendamustine and N-methyl-2-pyrrolidone as a solvent. In another embodiment, a a ready to use parenteral composition consists essentially of, or consists of, about 25 mg/ml to about 100 mg/ml of bendamustine and N-methyl-2-pyrrolidone as a solvent.

Another embodiment of the present invention relates to a ready to use parenteral composition comprising, consisting essentially of, or consisting of, per each ml of composition:
a) 25 mg of bendamustine hydrochloride and
b) N-methyl-2-pyrrolidone as a solvent.

Another embodiment of the present invention relates to ready to use parenteral composition comprising, consisting of, or consisting essentially of per each ml of composition:
a) 90 mg of bendamustine hydrochloride and
b) N-methyl-2-pyrrolidone as a solvent.

Another embodiment of the present invention relates to ready to use parenteral composition comprising, consisting essentially of, or consisting of, per each ml of composition:
a) 90 mg of bendamustine hydrochloride,
b) 0.1 to 0.5 ml diethylene glycol monoethyl ether, and
c) polar solvent selected from N-methyl-2-pyrrolidone (NMP) and polyethylene glycol.

Other embodiment of the present invention relates to process for the preparation of parenteral compositions of bendamustine comprising, consisting essentially of, or consisting of the steps of:
(a) adding a weighed quantity of bendamustine hydrochloride to diethylene glycol monoethyl ether and stirring until the bendamustine hydrochloride is dissolved completely,
b) optionally adding one or more pharmaceutically acceptable excipients to the solution of a)and stirring until the excipients are dissolved completely,
(c) filtering the solution and filling the filtered solution into vials,
(d) stoppering the vials, sealing the vials and storing the vials at 2-8° C.

Pharmaceutically acceptable excipients include bulking agents, solubilizers, buffers, pH adjustment aids, chelating agents, antioxidants, antibacterial preservatives and combinations thereof

Bulking agents include but are not limited to mannitol, lactose, sucrose, sodium chloride, trehalose, dextrose, starch, hydroxyethylstarch, cellulose, cyclodextrins, glycine, and mixtures thereof

Solubilizers include surface active agents, co-solvents, complexing agents and combinations thereof

Surface active agents include but are not limited to sorbitan fatty acid esters, polysorbates, poloxamers, oleoyl and linoleoyl polyoxylglycerides (such as Labrafil®), caprylocaproyl polyoxylglycerides (such as Labrasol®), Medium-chain triglycerides (such as Labrafac® lipophile), propylene glycol dicaprylocaprate (such as Labrafac° PG) andmixtures thereof.

Buffers include an acid or a base or a conjugate base or acid, respectively. Exemplary buffers include mixtures of weak acids and alkali metal salts (e.g., sodium, potassium) of the weak acids, such as acetate, citrate, tartarate, phosphate, benzoate and bicarbonate buffers and combinations thereof.

pH adjustment aids include but are not limited to tartaric acid, citric acid, malic acid, sodium chloride, potassium chloride, sodium hydroxide, potassium hydroxide, sodium carbonate, meglumine and combinations thereof

Chelating agents according includes but are not limited to ethylenetetraamineacetic acid, ethylenediaminetetraacetic acid (EDTA), and salts, derivatives and combinations thereof.

Antioxidants include but are not limited to ascorbic acid, sodium sulfite, sodium bisulfite and sodium metabisulfite and combinations thereof

Antibacterial preservatives include but are not limited to phenylmercuric nitrate, thiomersal, benzalkonium chloride, benzethonium chloride, phenol, cresol and chlorobutanol and combinations thereof.

Another embodiment of the present invention relates to process for the preparation of parenteral compositions of bendamustine hydrochloride comprising, consisting essentially of, or consisting of, the steps of:
a) adding a weighed quantity of bendamustine to N-methyl-2-pyrrolidone in a vessel and stirring until the bendamustine hydrochloride is dissolved completely,
b) adjusting final volume to 100% using N-methyl-2-pyrrolidone,
c) filtering the solution and filling the filtered solution into vials,
d) stoppering the vials, sealing the vials and storing the vials at 2-8° C.

Another embodiment of the present invention relates to process for the preparation of parenteral compositions of bendamustine hydrochloride comprising, consisting essentially of, or consisting of, the steps of:
a) adding a weighed quantity of bendamustine to a polar solvent and stirring until the bendamustine is dissolved completely,
b) adding a quantity of diethylene glycol monoethyl ether to the solution of step a),
c) adjusting the final volume to 100% batch size with polar solvent,
d) filtering the solution and filling the filtered solution into vials,
e) stoppering the vials, sealing the vials and storing the vials at 2-8° C.

Compositions of the present invention can preferably be diluted using 0.9% Sodium Chloride Injection, USP, or 2.5% Dextrose/ 0.45% Sodium Chloride Injection, USP before parenteral administration.

In yet another embodiment, the composition of the present invention is useful for the treatment of chronic lymphocytic leukemia and B-cell non-Hodgkin lymphoma.

### EXAMPLES

The following examples further describe and demonstrate particular embodiments within the scope of the present invention. The examples are given solely for illustration and are not to be construed as limitations as many variations are possible without departing from spirit and scope of the invention. It is obvious to those skilled in the art to find out the composition for other dosage forms and substitute the equivalent excipients as described in this specification or with the one known to the industry.

### Example 1

### Parenteral Compositions of Bendamustine

| Ingredients | Quantity per ml |
|---|---|
| Bendamustine HCl | 25 mg |
| Transcutol^{#} | q.s. to 1 ml |

Transcutol^{#}—diethylene glycol monoethyl ether

Brief Manufacturing Process:
1. weighed quantity of bendamustine was added to approximately 90% of transcutol in a vessel and stirred until the bendamustine dissolved completely,
2. the final volume was adjusted to 100% using transcutol,
3. the solution of step 2 was pre-filtered using 0.45μ sterile grade filters,
4. the pre-filtered solution of step 3 was filtered through 0.22μ sterile grade filters,
5. the filtered bulk solution of step 4 was filled into USP Type I amber glass vials,
6. the vials of step 5 were stoppered, sealed and stored at 2 to 8° C.

### Example 2

Parenteral Compositions of Bendamustine

| Ingredients | Quantity per ml |
|---|---|
| Bendamustine HCl | 90 mg |
| Transcutol^{#} | q.s to 1 ml |

Transcutol^{#}—diethylene glycol monoethyl ether

Brief Manufacturing Process:
1. weighed quantity of bendamustine was added to approximately 90% of transcutol in a vessel and stirred until the bendamustine dissolved completely,
2. the final volume was adjusted to 100% batch size using transcutol,
3. the solution of step 2 was pre-filtered using 0.45μ sterile grade filters,
4. the pre-filtered solution of step 3 was filtered through 0.22μ sterile grade filters,
5. the filtered bulk solution of step 4 was filled into USP Type I amber glass vials,
6. the vials of step 5 were stoppered, sealed and stored at 2 to 8 ° C.

### Example 3

Parenteral Compositions of Bendamustine

| Ingredients | Quantity per ml |
|---|---|
| Bendamustine HCl | 25 mg |
| N-methyl-2-pyrrolidone | q.s. to 1 ml |

Brief Manufacturing Process:
1. weighed quantity of bendamustine was added to approximately 90% of N-methyl-2-pyrrolidone in a vessel and stirred until the bendamustine dissolved completely,
2. the final volume was adjusted to 100% using N-Methyl-2-pyrrolidone,
3. the solution of step 2 was pre-filtered using 0.45μ sterile grade filters,
4. the pre-filtered solution of step 3 was filtered through 0.22μ sterile grade filters,
5. the filtered bulk solution of step 4 was filled into USP Type I amber glass vials,
6. the vials of step 5 were stoppered, sealed and stored at 2 to 8 ° C.

### Example 4

Parenteral Compositions of Bendamustine

| Ingredients | Quantity per ml |
|---|---|
| Bendamustine HCl | 90 mg |
| Transcutol^{#} | q.s to 0.1 ml |
| N-methyl-2-pyrrolidone | q.s to 1 ml |

Transcutol^{#}—diethylene glycol monoethyl ether

Brief Manufacturing Process:
1. weighed quantity of bendamustine was added to approximately 75% of N-methyl-2-pyrrolidone in a vessel and stirred until the bendamustine dissolved completely,
2. transcutol was added to the solution of step 1 and stirred,
3. the final volume was adjusted to 100% batch size using N-methyl-2-pyrrolidone,
4. the solution of step 3 was pre-filtered using 0.45μ sterile grade filters,
5. the pre-filtered solution of step 4 was filtered through 0.22μ sterile grade filters,
6. the filtered bulk solution of step 5 was filled into USP Type I amber glass vials,
7. the vials of step 6 were stoppered, sealed and stored at 2 to 8 ° C.

### Example 5

Parenteral Compositions of Bendamustine

| Ingredients | Quantity per ml |
|---|---|
| Bendamustine HCl | 90 mg |
| Transcutol^{#} | q.s to 0.1 ml |
| Polyethylene glycol | q.s to 1 ml |

Transcutol^{#}—diethylene glycol monoethyl ether

Brief Manufacturing Process:
1. weighed quantity of bendamustine was added to approximately 75% of polyethylene glycol in a vessel and stirred until the bendamustine dissolved completely,
2. transcutol was added to the solution of step 1 and stirred,
3. the final volume was adjusted to 100% batch size using polyethylene glycol,
4. the solution of step 3 was pre-filtered using 0.45μ sterile grade filters,
5. the pre-filtered solution of step 4 was filtered through 0.22μ sterile grade filters,
6. the filtered bulk solution of step 5 was filled into USP Type I amber glass vials,

17. the vials of step 6 were stoppered, sealed and stored at 2 to 8 ° C.

### Example 6

Parenteral Compositions of Bendamustine

| Ingredients | Quantity per ml |
|---|---|
| Bendamustine HCl | 90 mg |
| N-methyl-2-pyrrolidone | q.s. to 1 ml |

The manufacturing process is same as that of Example 3.

Stability Studies

The composition prepared according to the example 4 was stored at 2-8° C. and was tested for impurities at specific intervals. The results are as follows:

| Test Parameters | Initial | 1 Month | 2 Month | 3 Month |
|---|---|---|---|---|
| Assay | 99.7 | 100.3 | 99.7 | 98.5 |
| Related compounds | | | | |
| HP1impurity | 0.023 | 0.037 | 0.045 | 0.070 |

1. A ready to use parenteral composition comprising:
a) bendamustine or its pharmaceutically acceptable salt,
b) diethylene glycol monoethyl ether, and
c) a polar solvent selected from N-methyl-2-pyrrolidone (NMP), and polyethylene glycol.

2. The ready to use parenteral composition of claim 1, wherein the polar solvent comprises N-methyl-2-pyrrolidone

3. The ready to use parenteral composition of claim 1, comprising 0.1 ml to 0.5 ml of diethylene glycol monoethyl ether per ml of ready to use parenteral composition.

4. The ready to use parenteral composition of claim 1, comprising about 25 mg/ml to about 100 mg/ml of bendamustine.

5. The ready to use parenteral composition of claim 4, comprising 90 mg of bendamustine hydrochloride.

6. The ready to use parenteral composition of claim 1, further comprising a pharmaceutically acceptable excipient.

7. The ready to use parenteral composition of claim 6, wherein the pharmaceutically acceptable excipient comprises a solubilizer, a buffer, a pH adjustment aid, a chelating agent, an antioxidant, an antibacterial preservative, or a and combination thereof

8. A process for the preparation of a composition of claim 1, comprising:
a) adding a weighed quantity of bendamustine hydrochloride to diethylene glycol monoethyl ether and stirring until the bendamustine hydrochloride is dissolved completely,
b) adjusting the final volume up to 100% using N-methyl-2-pyrrolidone or polyethylene glycol,
c) filtering the solution of b) and filling the filtered solution into vials,
d) stoppering the vials, sealing the vials and storing the vials at 2-8° C.
